(19) United States
(12) Patent Application Publication
(10) Pub. No.: US 2024/0076604 A1
(43) Pub. Date: Mar. 7, 2024

(54) LARGE SCALE BIOREACTOR SYSTEM AND METHOD

(71) Applicant: AMGEN INC., Thousand Oaks, CA (US)

(72) Inventors: Gregory S. Stimpfl, Thousand Oaks, CA (US); Oliver Kaltenbrunner, Moorpark, CA (US); Mark O. Brothers, Thousand Oaks, CA (US); David C. Hogenson, Thousand Oaks, CA (US); Thomsen P. Sawicky, Thousand Oaks, CA (US); Min Kyu Choi, Thousand Oaks, CA (US); Jeffrey T. Ranney, Thousand Oaks, CA (US); John C. Roseland, Thousand Oaks, CA (US)

(21) Appl. No.: 18/242,116

(22) Filed: Sep. 5, 2023

Related U.S. Application Data
(60) Provisional application No. 63/404,033, filed on Sep. 6, 2022.

(51) Int. Cl. C12M 1/34; C12M 1/00

(52) U.S. Cl. CPC C12M 41/40; C12M 23/28; C12M 23/40; C12M 29/04; C12M 29/06; C12M 29/10; C12M 39/00

(57) ABSTRACT

A large scale bioreactor system includes a stainless steel large scale bioreactor having at least one valve assembly, and an aseptic connector assembly coupled to the at least one valve assembly of the bioreactor. A perfusion device includes an Alternating Tangential Filtration assembly with an autoclaved valve assembly coupled to the aseptic connector assembly, and the aseptic connector assembly includes one of a triclamp aseptic connector or a hose assembly. Single use feed containers include an aseptic connector assembly.

### CROSS-REFERENCE TO RELATED APPLICATIONS

This application claims priority to and the benefit of the filing date of provisional U.S. Patent Application No. 63/404,033, filed on Sep. 6, 2022. The entire contents of the provisional application are hereby expressly incorporated herein by reference.

### FIELD OF DISCLOSURE

The present disclosure generally relates to large scale bioreactor systems and, more particularly, to integration of perfusion devices and/or single use feed containers with large scale bioreactors and associated management of pressure.

### BACKGROUND

The use of perfusion in a cell culture bioreactor allows improved performance compared to a traditional fed batch cell culture process. Perfusion allows continuous addition of nutrients to the cell culture and continuous removal of metabolic byproducts. In fed batch mode, the tank volume limits the cell culture process, and all byproducts are contained inside the bioreactor until a harvest.

Traditionally, perfusion has been applied to small scale bioreactors, for example, up to 2000 L in working volume. Pressure realized by the perfusion device is greater on a large scale bioreactor due to an increased height of liquid (static head) above the perfusion device when installed in a traditional location near a bottom of a side of the large scale stainless steel bioreactor. In addition, perfusion devices that contain single use components are particularly sensitive to pressure due to the low pressure rating of the single use components.

### SUMMARY

In accordance with a first aspect, a large scale bioreactor system comprises a stainless steel large scale bioreactor having at least one valve assembly, and an aseptic connector assembly coupled to the least one valve assembly of the bioreactor. A perfusion device including an Alternating Tangential Filtration (ATF) assembly with an autoclaved valve assembly is coupled to the aseptic connector assembly, and the aseptic connector assembly includes one of a triclamp aseptic connector or a hose assembly.

In accordance with a second aspect, a large scale bioreactor system comprises a stainless steel large scale bioreactor having a side, and an autoclaved valve assembly coupled to the side of the bioreactor. At least one aseptic connector is coupled to the autoclaved valve assembly, and an irradiated single use perfusion device is coupled to the at least one aseptic connector.

In accordance with yet another aspect, a large scale bioreactor system comprises a stainless steel large scale bioreactor having at least one valve assembly, and a single use adapter assembly including a wye connector assembly coupled to the at least one valve assembly. A plurality of single use perfusion devices are connected to the single use adapter assembly, enabling multiple perfusion units to be coupled to the bioreactor without having to steam-in-place the bioreactor upon coupling multiple single use perfusion units.

In accordance with yet another aspect, a large scale bioreactor system comprises a stainless steel large scale bioreactor, and at least one stainless steel transfer panel having a plurality of inputs coupled to the bioreactor. A plurality of single use feed containers are coupled to the at least one stainless steel transfer panel at a working level of the bioreactor. Another aspect is that the stainless steel large scale bioreactor may be installed in a pit depression in a floor to facilitate easy access to a probe belt for operations. Another aspect is that the fully-closed stainless steel large scale bioreactor may be installed in an uncontrolled space and operate exclusively at the probe belt and a local addition panel. So configured, the size of the controlled space is reduced, leading to lower operating cost for a manufacturing facility housing the bioreactor.

In accordance with yet another aspect, a method of integrating at least one single use perfusion device with a stainless steel large scale bioreactor comprises coupling one of: (1) a connector assembly to at least one valve assembly of a stainless steel large scale bioreactor; or (2) an autoclaved valve assembly to a side of the stainless steel large scale bioreactor at a higher elevation to reduce pressure from the static head of the liquid. The method further comprises coupling one of: (1) an autoclaved valve assembly of an ATF assembly of a single use perfusion device to the connector assembly; or (2) an irradiated single use perfusion device to the autoclaved valve assembly. The method still further comprises managing pressure of the single use perfusion device via at least one pressure sensor of the single use perfusion device and automatically reducing one or more of a flow rate or a pressure in the single use perfusion device upon detecting a pressure greater than a safe limit pressure by a control system.

In some embodiments of any aspect, the stainless steel large scale bioreactor is configured to hold a volume of greater than 2,000 L, such as, e.g., a volume in the range of greater than 2,000 L to 20,000 L, such as, e.g., a volume in the range of 10,000 L to 20,000 L.

### BRIEF DESCRIPTION OF THE DRAWINGS

It is believed that the disclosure will be more fully understood from the following description taken in conjunction with the accompanying drawings. Some of the drawings may have been simplified by the omission of selected elements for the purpose of more clearly showing other elements. Such omissions of elements in some drawings are not necessarily indicative of the presence or absence of particular elements in any of the example embodiments, except as may be explicitly delineated in the corresponding written description. Also, none of the drawings is necessarily to scale.

FIG. 1 is a schematic representation of a steam-in-place (SIP) bioreactor (BRX) having a valve assembly of a large scale stainless steel bioreactor system according to one aspect of the present disclosure, showing how the steam-in-place bioreactor can be steamed in place without a perfusion device or a feed container connected to the valves;

FIG. 2 is a schematic representation of the steam-in-place bioreactor of FIG. 1 having an aseptic connector assembly coupled to the valve assembly of the steam-in-place bioreactor;

FIG. 3 is another schematic representation of the steam-in-place bioreactor of FIG. 2 with the connector assembly coupled to the valve assembly as can be steamed in place, independent of the operation of the steam-in-place bioreactor;

FIG. 4 is a schematic representation of the steam-in-place bioreactor of FIG. 2 or FIG. 3, with a single use (SU) perfusion device or a reusable perfusion device, including an Alternating Tangential Filtration (ATF) assembly coupled to the connector assembly using the aseptic connector;

FIG. 5 is a close-up view of the perfusion device of FIG. 4 and associated valve assembly after they are cleaned out of place (COP) and assembled prior to autoclaving in preparation for connection to the steam-in-place bioreactor;

FIG. 6 is another large scale bioreactor system according to another aspect of the present disclosure, the large scale bioreactor system having one of a plurality of autoclaved valve assemblies coupled to a large scale stainless steel bioreactor and an irradiated single use perfusion device aseptically coupled to the autoclaved valve assemblies using an aseptic connector;

FIG. 7 is another large scale bioreactor system according to another aspect of the present disclosure, the large scale bioreactor system including a hose assembly coupling at least one valve assembly of the stainless steel large scale bioreactor to an autoclaved perfusion device, such as a single use perfusion device or a reusable autoclaved perfusion device, including an ATF assembly;

FIG. 8 is a schematic representation of a clean-in-place bioreactor having at least one valve assembly, having an ability to clean the at least one valve assembly in place with the clean-in-place bioreactor;

FIG. 9 is another large scale bioreactor system according to another aspect of the present disclosure, the large scale bioreactor system including a large scale stainless steel bioreactor having at least one valve assembly and a wye assembly coupled to the at least one valve assembly to enable the use of multiple perfusion devices on a single port;

FIG. 10 is a schematic representation of multiple wye assemblies coupled to multiple valve assemblies, enabling multiple single use feed containers to be coupled to the wye assemblies, and ultimately the large scale stainless steel bioreactor, without having to steam-in-place the valve assembly multiple times;

FIG. 11 is a schematic representation of a large scale bioreactor system having a large scale stainless steel bioreactor, the large scale bioreactor system having one of a plurality of autoclaved valve assemblies coupled to a top portion of the large scale stainless steel bioreactor and an irradiated perfusion device aseptically coupled to at least one autoclaved valve assemblies;

FIG. 12 is a schematic representation of a large scale stainless steel bioreactor of any one of the large scale bioreactor systems of the present disclosure at least partially disposed in a pit to enable efficient operation at a probe belt and transfer panel; and

FIG. 13 is a schematic representation of a large scale stainless steel bioreactor having a transfer panel coupled to the bioreactor, allowing multiple feed containers to be coupled to the stainless steel bioreactor at a working level.

### DETAILED DESCRIPTION

Generally, a large scale bioreactor system is disclosed. The large scale bioreactor system comprises a stainless steel large scale bioreactor having at least one valve assembly, an aseptic connector assembly coupled to the at least one valve assembly of the bioreactor, and a single use or reusable perfusion device coupled to the aseptic connector assembly. The single use perfusion device includes an perfusion filter assembly with an autoclaved valve assembly coupled to the aseptic connector assembly. So configured, a new perfusion device may be installed while the stainless steel large scale bioreactor is running a cell culture by repeating a steam-in-place of the aseptic connector assembly, or through the use of an aseptic connector valve assembly.

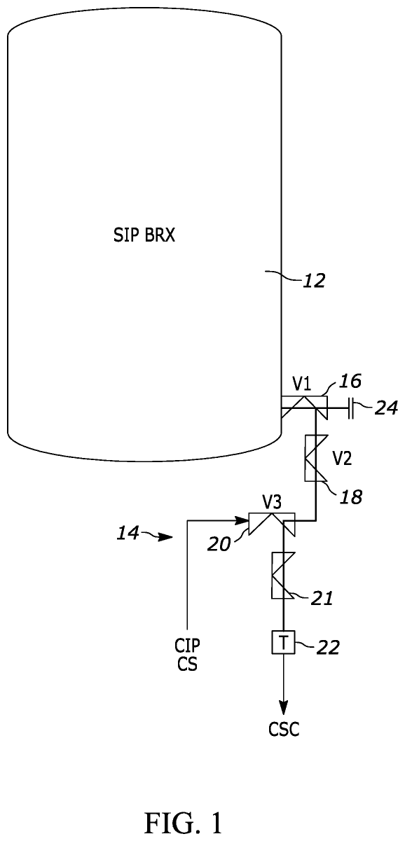

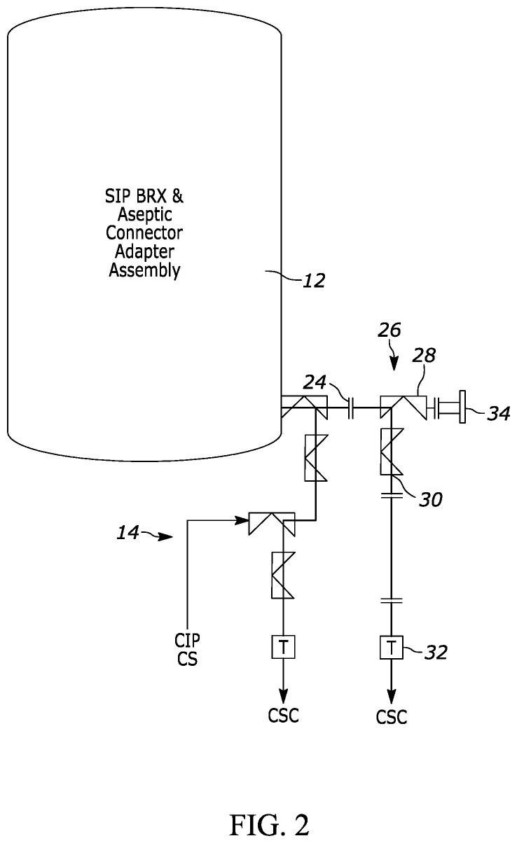

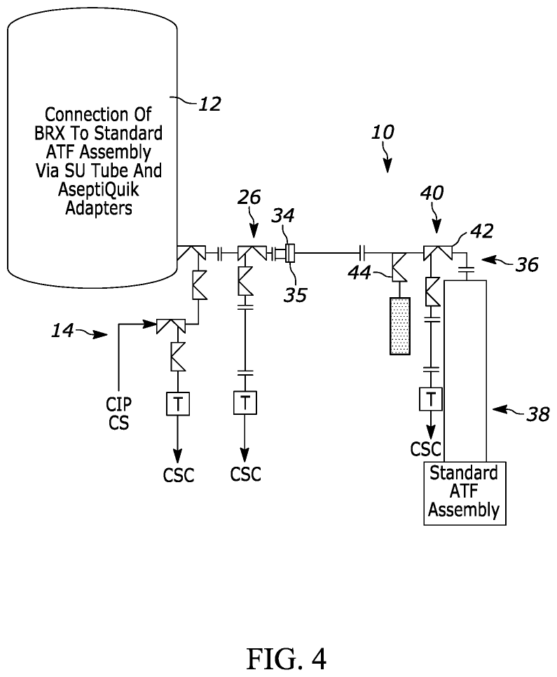

Referring now to FIGS. 1-5, schematic representations of a large scale bioreactor system 10 (FIG. 4) according to several aspects of the present disclosure are depicted. As depicted in FIG. 1, the large scale bioreactor system 10 includes a stainless steel large scale bioreactor 12 having at least one valve assembly 14. In these examples, the stainless steel large scale bioreactor 12 is a steam-in-place SIP bioreactor BRX with large scale capacity. For example, the large scale bioreactor 12 is configured to hold a volume of fluid of up to 10,000 L or more in one example. The at least one valve assembly 14 of the steam-in-place large scale bioreactor 12 enables the steam-in-place large scale bioreactor 12 to be completely sterilized before perfusion begins. Specifically, the at least one valve assembly 14 includes a first valve 16, a second valve 18, a third valve 20 disposed downstream from each of the first and second valves 16, 18, and a fourth valve 21. Steam, such as clean steam CS, flows into the third valve 20, upwardly into the first and second valves 16, 18, and to a port 24. Additionally, after flowing into the third valve 20, the steam also flows downwardly into the fourth valve 21 and through a steam trap 22 in which clean steam condensate CSC is released, for example, to sterilize the bioreactor 12 and create a steam sterilized aseptic environment. In one example, when a SIP cycle is finished, condensate, such as clean steam condensate CSC, drains from the steam trap 22. In addition, process waste PW flows out of the third and fourth valves 20, 21 during cleaning, emptying into a drain, as is understood by those having ordinary skill. After use, the steam-in-place bioreactor 12 is cleaned in-place and the steam-in-place process of sterilization occurs again before any further use. The at least one valve assembly 14 also includes the port 24 for coupling to an aseptic connector valve assembly 26, such as an autoclaved aseptic connector valve assembly 26, as depicted in FIG. 2, or another device, as explained more below.

Referring now to FIG. 2, the aseptic connector valve assembly 26 is coupled to the at least one valve assembly 14. Specifically, and in this example, the aseptic connector valve assembly 26 is coupled to the port 24 of the at least one valve assembly 14 and includes a first valve 28 and a second valve 30 disposed downstream from the first valve 28. A steam trap 32 is disposed further downstream from the second valve 30. In this example, the autoclaved aseptic connector valve assembly 26 also includes a triclamp aseptic connector 34 coupled to the first valve 28. The aseptic connector valve assembly 26 enables sterilized connectivity of a single use perfusion device or a reusable perfusion device to the large scale bioreactor 12. The triclamp aseptic connector 34 is configured to couple to a perfusion device, as explained more below. As will be understood, and in one example, the triclamp aseptic connector 34, and the first and second valves 28, 30 may be preassembled and autoclaved before connecting to the at least one valve assembly 14 for steam sterilization, for example.

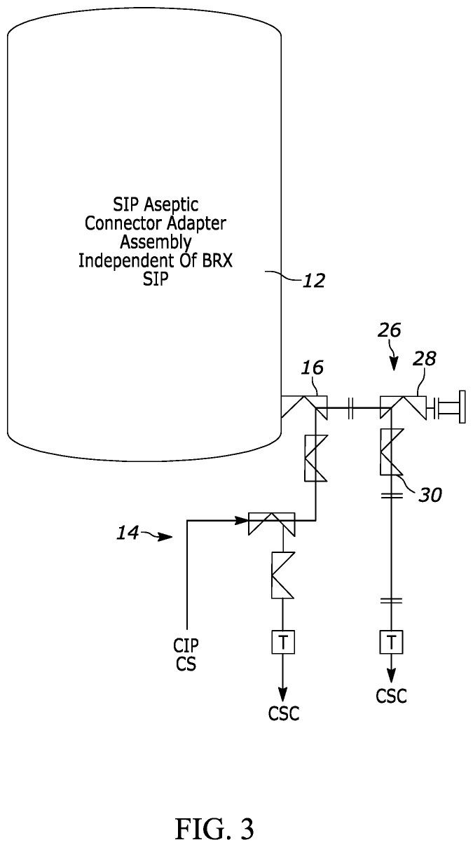

Referring now to FIG. 3, the large scale bioreactor 12 having the at least one valve assembly 14 and the aseptic connector valve assembly 26 coupled to the at least one valve assembly 14 is again depicted. In this example, however, the aseptic connector valve assembly 26 is sterilized independent of the steam-in-place large scale bioreactor 12. Specifically, if during a cell culture run in the large scale bioreactor 12, it is determined that a perfusion device needs to be serviced and/or replaced, for example, the run of the large scale bioreactor 12 may continue. This is because the aseptic connector valve assembly 26 is able to steam the first valve 28 and the second valve 30, for example, and all other parts of the aseptic connector valve assembly 26 to provide a sterilized connector assembly for coupling to the repaired and/or new perfusion device.

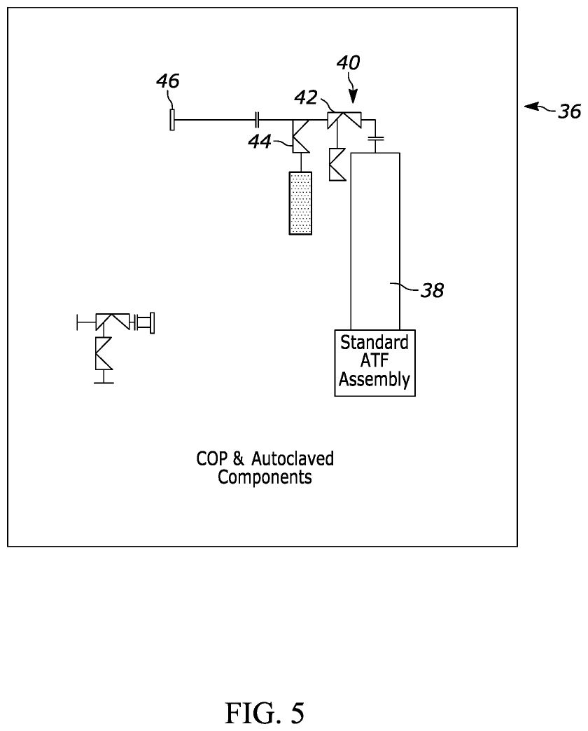

Referring now to FIG. 4, a schematic representation of the large scale bioreactor system 10 according to an aspect of the present disclosure is depicted. More specifically, the stainless steel large scale bioreactor 12 having the at least one valve assembly 14 and the aseptic connector valve assembly 26 coupled to the at least one valve assembly 14 is depicted. In addition, a perfusion device 36, which may be a reusable perfusion device or a single use perfusion device, is coupled to the triclamp aseptic connector 34 of the aseptic connector valve assembly 26. As depicted in both FIG. 4 and the close up view of the perfusion device 36 in FIG. 5, the perfusion device 36 includes an Alternating Tangential Filtration (ATF) assembly 38 with an autoclaved valve assembly 40 operatively coupled to the aseptic connector 34 of the aseptic connector valve assembly 26. So configured, the large scale bioreactor system 10 enables connectivity of the perfusion device 36, such as a reusable perfusion device or a single use perfusion device, to the stainless steel large scale bioreactor 12 without additional steam sterilization.

As also depicted in FIG. 5, the autoclaved valve assembly 40 of the perfusion device 36 includes a first valve 42 and a second valve 44 disposed downstream from the first valve 42. In this example, the autoclaved valve assembly 40 is cleaned out of place (COP) and assembled prior to autoclaving in preparation for connection to the steam-in-place bioreactor 12. Generally, COP denotes systems and equipment, such as the autoclaved valve assembly 40 in this example, which are one or more of disassembled, relocated, or specialty treated to clean and sanitize. Said another way, COP is defined as a method of cleaning equipment items by removing them from their operational area and taking them to a designated cleaning station for cleaning. In addition, an aseptic connector 46 is operatively coupled to the first valve 42 and directly coupled to the triclamp aseptic connector 34 of the aseptic connector assembly 26, as depicted in FIG. 4. The aseptic connector assembly 26 allows connectivity between the single use perfusion device 36 and the stainless steel large scale bioreactor 12 and provides an ability to restream on subsequent perfusion devices during a single run of the bioreactor 12. Said another way, with this configuration, the perfusion device 36 and/or a new perfusion device may be installed while the stainless steel large scale bioreactor 12 is running a cell culture, by repeating the steam-in-place or by using the aseptic connector assembly 26, for example.

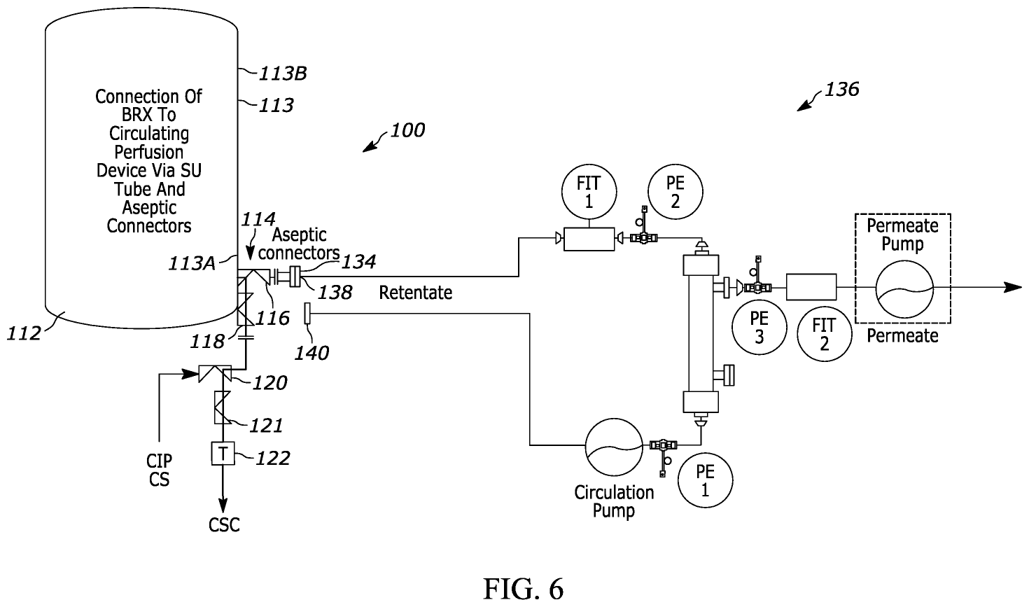

Referring now to FIG. 6, another large scale bioreactor system 100 is depicted. Like the large scale bioreactor system 10 of FIGS. 1-5, the large scale bioreactor system also includes a stainless steel large scale bioreactor 112 having a side 113 and a first valve assembly 114, such as an autoclaved valve assembly 114, coupled to the side 113 of the stainless steel large scale bioreactor 112. The autoclaved valve assembly 114 includes a first valve 116, a second valve 118 downstream from the first valve 116, a third valve 120 downstream from both the first and second valves 116, 118, and a fourth valve 121 downstream from the third valve 120. Again like the valve assembly 14 of the system 10 of FIGS. 1-5, steam, such as clean steam CS, flows into the third valve 120, upwardly to the first and second valves 116, 118 and to the port 134. Additionally, after flowing into the third valve 120, steam flows downwardly through the fourth valve 121 and through a steam trap 122 to sterilize the bioreactor 112 and create a steam-in-place process of sterilization before further use, for example. In this large scale bioreactor system 100, however, there is only at least one aseptic connector 134 coupled to the autoclaved valve assembly 114, such as the first valve 116 of the autoclaved valve assembly 114, and not a second valve assembly, like the aseptic connector assembly 26 of the system 10 of FIGS. 1-5. This same autoclaved valve assembly 114 enables connection via the at least one aseptic connector 134 of the autoclaved valve assembly 114 to a factory assembled and irradiated perfusion device 136, which may be a single use perfusion device or a reusable perfusion device, for example. More specifically, the irradiated perfusion device 136 includes a first aseptic connector 138 that is coupled to the at least one aseptic connector 134, as depicted in FIG. 6. The irradiated perfusion device 136 also includes a second aseptic connector 140 that is configured to be coupled to a second autoclaved valve assembly (not shown), such as a second valve assembly, which is also configured to be directly coupled to the side 113 of the large scale stainless steel bioreactor 112 on a different valve assembly analogous to the valve assembly 114. As a result, multiple perfusion devices are able to be aseptically coupled to the large scale stainless steel bioreactor 112 without additional steam sterilization.

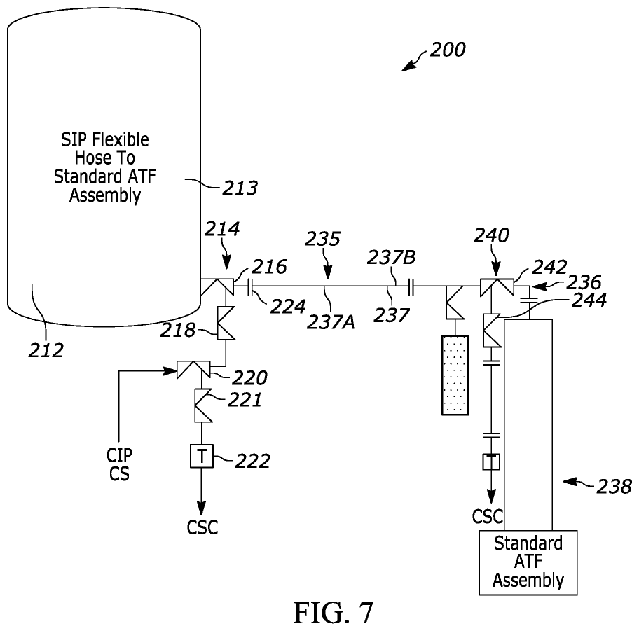

Referring now to FIG. 7, a schematic view of another large scale bioreactor system 200 of the present disclosure is depicted. Like the large scale bioreactor system 10 of FIG. 4, for example, the large scale bioreactor system 200 includes a stainless steel large scale bioreactor 212 having a side wall 213 and at least one valve assembly 214 disposed on the side wall 213. Again like the large scale bioreactor system 10 of FIG. 4, the same valve assembly 214 of the large scale bioreactor 200 of FIG. 7 enables connection of an autoclaved perfusion device and cleaning-in-place of the valve assembly 214, but without the use of an aseptic connector, such as the aseptic connector 34 of FIG. 4. Instead, a hose assembly is used as an alternate connector to couple the stainless steel large scale bioreactor 212 to the autoclaved perfusion device, as explained more below.

More specifically, and as depicted in FIG. 7, the stainless steel large scale bioreactor 212 of the large scale bioreactor system 200 is also a steam-in-place bioreactor with large scale capacity. For example, the large scale bioreactor 212 is configured to hold a volume of fluid of up to or greater than 10,000 L in one example. The at least one valve assembly 214 of the steam-in-place large scale bioreactor 212 enables the steam-in-place, stainless steel large scale bioreactor 212 to be completely sterilized when perfusion begins. Specifically, and like the at least one valve assembly 14 of the bioreactor system 10 of FIG. 4, the at least one valve assembly 214 includes a first valve 216, a second valve 218, and a third valve 220 disposed downstream from each of the first and second valves 216, 218. A fourth valve 221 may also be disposed downstream from the third valve 220, as depicted. Steam, such as clean steam CS, again flows into the third valve 220, upwardly to the first and second valves 116, 118 and to a port 224. Additionally, after flowing into the third valve 220, steam also flows downwardly into the fourth valve 221 and through a steam trap 222 to sterilize the bioreactor 212 and create a steam sterilized aseptic environment. In addition, process waste PW again may flow out of the third and fourth valves 220, 221 during cleaning, emptying into a drain, as is understood by those having ordinary skill. After use, the steam-in-place bioreactor 212 is cleaned in-place and the steam-in-place process of sterilization occurs again before any further use. The at least one valve assembly 214 also includes the port 224 for coupling to a hose assembly 235, which in turn couples to a perfusion device or another device, as explained more below.

The hose assembly 235 includes a hose body 237 having a first end 237A and a second end 237B. The first end 237A is removably coupled to the port 224 of the at least one valve assembly 214 and the second end 237B is coupled to an autoclaved perfusion device 236, which is functionally equivalent to the perfusion device 36 of the system 10 of FIG. 4, for example, but without a single-use connector, such as the single-use connector 46 of FIG. 5. The autoclaved perfusion device 236 may include a single-use perfusion device or a reusable perfusion device and still fall within the scope of the present disclosure. The autoclaved perfusion device 236 includes an ATF assembly 238 with an autoclaved valve assembly 240 coupled to the second end 237B of the hose body 237. The autoclaved valve assembly 240 of the autoclaved perfusion device 236 includes a first valve 242, and a second valve 244 disposed downstream from the first valve 242. The second end 237B of the hose body 237 is operatively coupled to a connector of the first valve 242. The hose assembly 235 allows connectivity between the autoclaved device 236 and the stainless steel large scale bioreactor 212 and provides an ability to restream on subsequent perfusion devices during a single run of the stainless steel large scale bioreactor 212.

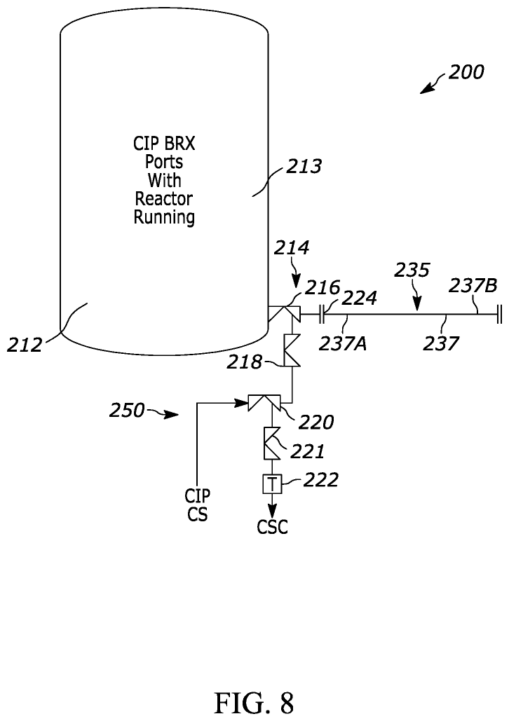

More specifically, and referring now to FIG. 8, the hose assembly 235 may be removed from connection to the perfusion device 236 and coupled to another one of multiple ports 224 on the same large scale bioreactor 212. A clean-in-place valve assembly 250 allows the at least one valve assembly 214 to be cleaned, which enables another perfusion device, such as a single use perfusion device, to be aseptically coupled to the stainless steel large scale bioreactor 212 via the hose assembly 235. In this example, a clean-in-place steam supply of the clean-in-place valve assembly 250 upwardly flows into the third valve 220 and the second valve 218 of the at least one valve assembly 214, up through the first valve 216, to port 224 and through the hose assembly 235. In addition, the clean-in-place steam supply also downwardly flows through the fourth valve 221 downstream from the third valve 220. In this way, each of the first, second, third and fourth valves 216, 218, 220 and 221 of the at least one valve assembly 214 and the port 224 are cleaned. While this description refers to the at least one valve assembly 214, it will be understood that the at least one valve assembly 214 may include multiple valve assemblies (not shown), and the clean-in-place steam supply of the clean-in-place valve assembly 250 may also flow through each of the valves and ports of another one of the multiple valve assemblies, allowing multiple valve assemblies to be cleaned.

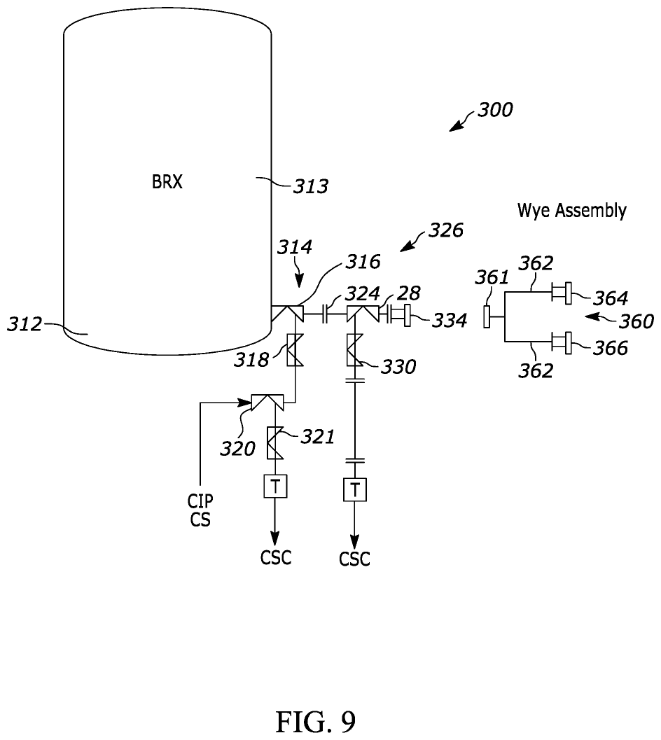

Referring now to FIG. 9, another schematic representation of another large scale bioreactor system 300 of the present disclosure is depicted. The large scale bioreactor system 300 includes a stainless steel large scale bioreactor 312 having a side 313 and at least one valve assembly 314 coupled to the side 313 and an autoclaved valve assembly 326 coupled to the at least one valve assembly 314. In one example, the stainless steel large scale bioreactor 312 is again a steam-in-place bioreactor with large scale capacity. For example, the large scale bioreactor 312 is configured to hold a volume of fluid of up to or greater than 10,000 L in one example. The at least one valve assembly 314 of the steam-in-place large scale bioreactor 312 enables the steam-in-place large scale bioreactor 312 to be completely sterilized when perfusion begins. Specifically, and like the at least one valve assembly 14 of FIG. 4, for example, the at least one valve assembly 314 includes a first valve 316, a second valve 318, a third valve 320 disposed downstream from each of the first and second valves 316, 318, and a fourth valve 321 disposed downstream from the third valve 320, as depicted. Steam, again such as clean steam CS, flows into the third valve 320 and upwardly into the second and first valves 318, 316 and a port 324 to sterilize the at least one valve assembly 314 and the bioreactor 312. In addition, after flowing through the third valve 320 steam also flows downwardly through the fourth valve 321 and through a steam trap 322 to sterilize the bioreactor 312 and create a steam sterilized aseptic environment. In addition, process waste PW flows out of the fourth valve 321 during cleaning, emptying into a drain, as is understood by those having ordinary skill. After use, the steam-in-place bioreactor 312 is cleaned in-place and the steam-in-place process of sterilization occurs again before any further use. The at least one valve assembly 314 again also includes the port 324 for coupling to the autoclaved valve assembly 326 and an aseptic connector 334.

Unlike the other bioreactor systems 10, 100, and 200, the bioreactor system 300 includes an adapter assembly 360, such as a single use adapter assembly and/or a wye assembly, which is configured to be and/or is coupled to the aseptic connector 334, as also depicted in FIG. 9. So configured, the wye assembly 360 is coupled to the at least one valve assembly 314 via the aseptic connector 334 in this example.

The adapter assembly 360, such as the wye assembly 360, includes a connector 361 that is directly coupled to the aseptic connector 334 (which is ultimately coupled to the port 324), a pair of tubes 362 outwardly extending from the connector 361, a first aseptic connector 364 coupled to one of the tubes 362, and a second aseptic connector 366 coupled to the other tube 362. Each of the first aseptic connector 364 and the second aseptic connector 366 of the wye assembly 360 is configured to be coupled to a reusable or single use perfusion device, such as any one of the foregoing perfusion devices 36, 136, and 236. This enables multiple perfusion devices to be operatively coupled to the stainless steel large scale bioreactor 312 via the port 324 of the valve assembly 314, for example, without having to steam-in-place the stainless steel large scale bioreactor 312 upon coupling multiple reusable perfusion devices or single use perfusion devices. In another example, the adapter assembly 360, such as the wye assembly 360, is a first wye assembly 360 and a second wye assembly (not shown) may be coupled to the first wye assembly. So configured, coupling one or more additional wye assemblies to the first wye assembly 360 enables more than two reusable or single use perfusion devices and multiple reusable or single use perfusion devices to be operatively coupled to the stainless steel large scale bioreactor 312, again without having to steam-in-place the stainless steel large scale bioreactor 312 upon coupling the multiple single use perfusion devices. Likewise, a greater number of perfusion devices can be connected to the large scale bioreactor 312 and each device does not require a distinct reactor port.

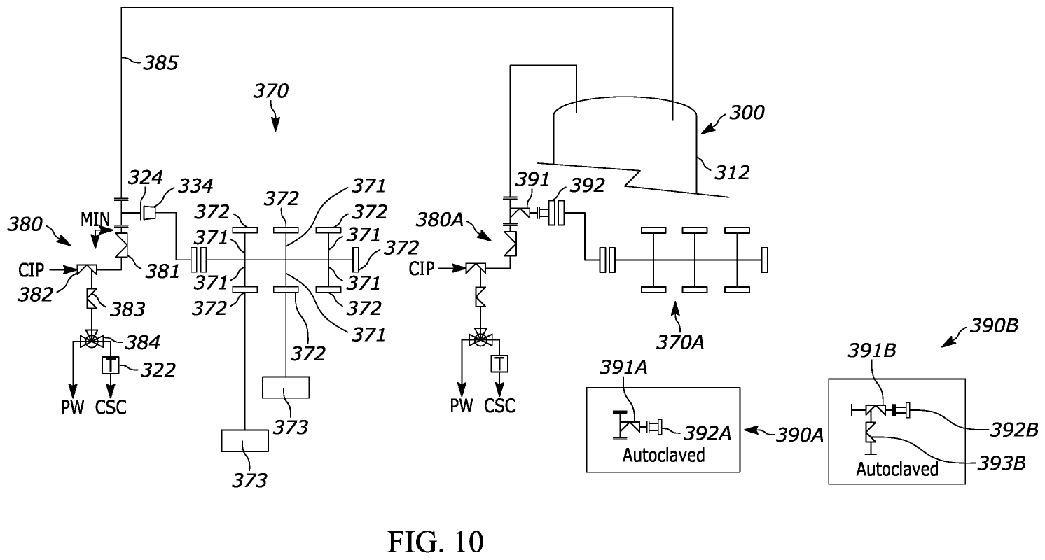

Referring now to FIG. 10, in another example, the large scale bioreactor system 300 is depicted and includes the large scale stainless steel bioreactor 312 that is configured to hold a volume of fluid of up to or greater than 10,000 L in one example. In this example, the at least one valve assembly of the large scale bioreactor system 300 includes the at least one valve assembly 380, which is configured to be coupled to a transfer panel (not depicted in FIG. 10), mounted locally or at a remote distance from the large scale stainless steel bioreactor 312 for the purpose of connecting and transferring feed liquids held in single use containers to the large scale stainless steel bioreactor 312. The valve assembly 380 is configured to enable steam sterilization of a transfer line 385 and connection to a single use feed container. Steam from the large scale bioreactor 300 passes out through at least one transfer line, such as the transfer line 385 in one example, and to a triclamp port 324 and the aseptic connector assembly 334, and through a first valve 381, a second valve 382, a third valve 383, and a fourth valve 384 and downwardly to the steam trap 322. As such, the entire line is rendered sterile up to the aseptic connector 334. Likewise, cleaning of the line is accomplished by supplying clean-in-place (CIP) solution through the first and second valves 381, 382 and through transfer line 385 to the large scale bioreactor 300. As depicted in FIG. 10, the ports 324 of the first and second valves 381, 382 are coupled to a single use adapter, such as the aseptic connector 334, which are designed to tolerate direct exposure to clean steam at sterilizing temperatures and pressures. As further depicted, the single use adapter, such as the aseptic connector assembly 334, in turn is coupled to a single use manifold 370. In this example, each single use manifold 370 includes eight or more arms 371 having inlets 372, each of which is configured to be coupled to a feed container 373, such as a single use feed container. In another example, the single use manifold 370 may include more or fewer than eight arms 371, such as four or six arms and still fall within the scope of the present disclosure. So configured, multiple feed containers are able to be operatively coupled to the stainless steel large scale bioreactor 312 without additional steaming and cleaning. One or more additional feed containers 373 can be connected using aseptic connectors 372 while the large scale stainless steel bioreactor 312 is in operation. In addition, one or more additional single use manifolds 370 may be connected to inlets 372 via the aseptic connector(s) on the first single use manifold assembly 370 to further increase the number of feed containers 373 that may be connected.

Referring again to FIG. 10, in the event that an aseptic connector is to be employed that cannot tolerate direct exposure to clean steam at sterilizing temperatures and pressures, an alternate configuration enables isolation of the aseptic connector from the steam. For example, one or more valve assemblies 390A and 390B may be prepared by coupling a valve or two valves, such as valve 391A of the valve assembly 390A, and first and second valves 391B, 393B of the valve assembly 390B, which may be any valve with a low hold up volume on the side to the steamed to an aseptic connector, such as an aseptic connector 392A or an aseptic connector 392B, and autoclaved. Each of valve assembly 390A and valve assembly 390B may be inserted in a valve assembly that may be installed on a local or remote transfer panel, such as at least one valve assembly 380A depicted in FIG. 10, by coupling with triclamp connectors, for example. From that point forward, the cleaning, steaming and operation are the same as that described above relative to FIG. 9. By using a single use manifold assembly with aseptic connector 392, a larger inner diameter, lower shear and higher flow rates are afforded than can be achieved using an aseptic connector that can be directly exposed to clean steam at sterilization temperatures and pressures. However, since the main transfer line 385 and the at least one valve assembly 380, 380A are the same, either type of aseptic connector may be used and a similar plurality of feed containers 373 can be connected. Alternatively, a single connection to the feed container 373 may be used when higher flow rates or lower shear are required.

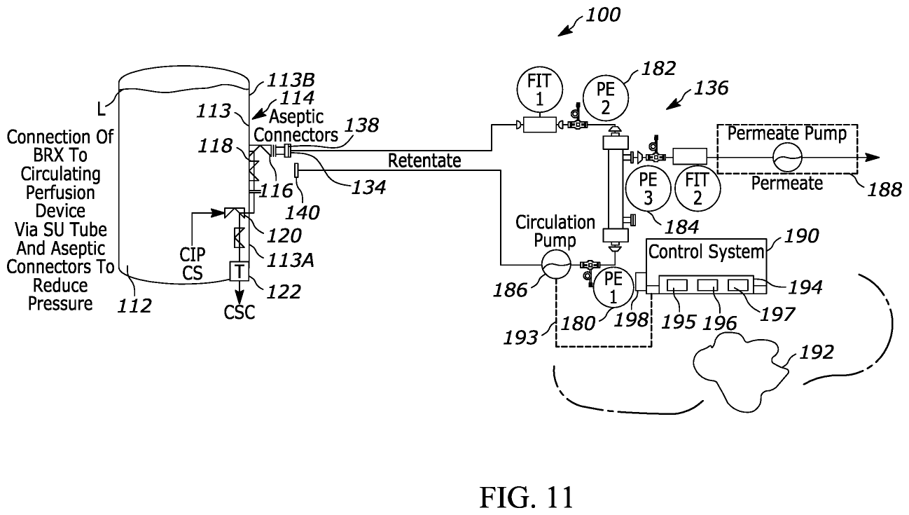

Referring now to FIG. 11, the bioreactor system 100 of FIG. 6 is again depicted and includes all of the same features of the bioreactor system 100 except for the location of the valve assembly 114 relative to the stainless steel large scale bioreactor 112. Specifically, the valve assembly 114 is mounted higher on the side 113 of the stainless steel large scale bioreactor 112, which in turns results in the perfusion device 136 also being mounted higher on the side 113 of the stainless steel large scale bioreactor 112. In particular, in this example, the side 113 of the bioreactor 112 includes a bottom end 113A and a top end 113B, and the valve assembly 114 is coupled to the side 113 of the bioreactor in a location closer to the top end 113B of the side 113 than the bottom end 113A, as depicted in FIG. 11. So configured, the height of liquid L disposed above the perfusion device 136, and therefore pressure, is reduced.

Generally, pressure realized by the single use perfusion device, such as the perfusion device 36, 136, 236, is greater on the stainless steel large scale bioreactor 12, 112, 212, 312, due to the increased height of liquid (static head) above the perfusion device 36, 136, 236 when installed in a typical location near a bottom side, such as the bottom end 113A, of the stainless steel large scale bioreactor 12, 112, 212, 312. Perfusion devices that include single use components are particularly sensitive to greater pressure due to the low pressure rating of the single use components, for example. Thus, by mounting the valve assembly 114 and, thus, the perfusion device 136 of FIG. 11, near the top end 113B of the bioreactor 112, pressure is reduced.

Still referring to FIG. 11, the perfusion device 136, such as the single use perfusion device, includes at least one pressure sensor 180, and may include additional pressure sensors. In this example, the perfusion device 136, such as an RFT single use perfusion device, includes a first pressure sensor 180, a second pressure sensor 182, and a third pressure sensor 184. In addition, the perfusion device 136 also includes a circulation pump 186 disposed upstream from the first pressure sensor 180, and a permeate pump 188 disposed downstream from the third sensor 184.

As further depicted in FIG. 11, a control system 190 for monitoring pressure of at least one perfusion device 136 is communicatively coupled via a network, such as a wireless network 192 or a wired connection 193, to the single use perfusion device 136. While the control system 190 is operatively coupled to the perfusion device 136, the control system 190 may also optionally be coupled to any one of the other foregoing described perfusion devices 36, 236 and still fall within the scope of the present disclosure. In one example, the control system 190 includes a computing device 194 having a memory 195, a processor 196, and a network interface 197. It will be understood that the computing device 194 may also include any known input, receiver and transmitter. In addition, the control system 190 further includes an alarm 198. So configured, the control system 190 monitors the pressure at one or more of the first, second and third pressure sensors 180, 182, 184. If the pressure approaches a pre-defined safe limit, the alarm 198 will be activated to notify users so the bioreactor system 100 may be investigated and evaluated. If the pressure being monitored by the control system 190 approaches still closer to the pre-defined safe limit, the control system 190 will automatically reduce the flow rate of fluid, and thus the pressure, to maintain a safe operating pressure for the bioreactor system 100. If the pressure being monitored by the control system 190 achieves the safe pressure limit, the control system 190 will stop the circulating pump 186, preventing damage to the perfusion device 136.

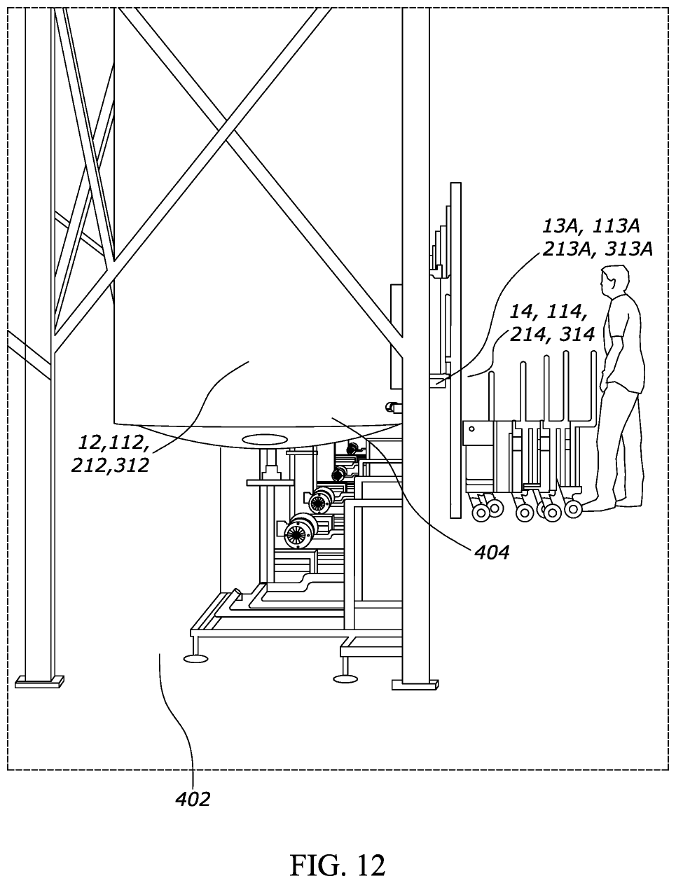

Referring now to FIG. 12, any one of the foregoing large scale stainless steel bioreactors 12, 112, 212, 312 may be disposed in a pit 402, as depicted in FIG. 12. In addition, the valve assembly 14, 114, 214, 314 is then mounted near the bottom end 13A, 113A, 213A, 313A of the side 13, 113, 213, 313 of the large scale stainless steel bioreactor 12, 112, 212, 312 and against a bottom weld seam 404 of a body of the bioreactor 12, 112, 212, 312. This enables the single use perfusion device 36, 136, 236 to be positioned higher, minimizing hold-up volume between the perfusion device 36, 136, 236 and the large scale stainless steel bioreactor 12, 112, 212, 312. Alternatively, by adjusting the depth of the pit, the pit 402 may be used to locate any one of the perfusion devices 36, 136, 236 higher on the side 13, 113, 213 of the bioreactor 12, 112, 212 to also reduce pressure.

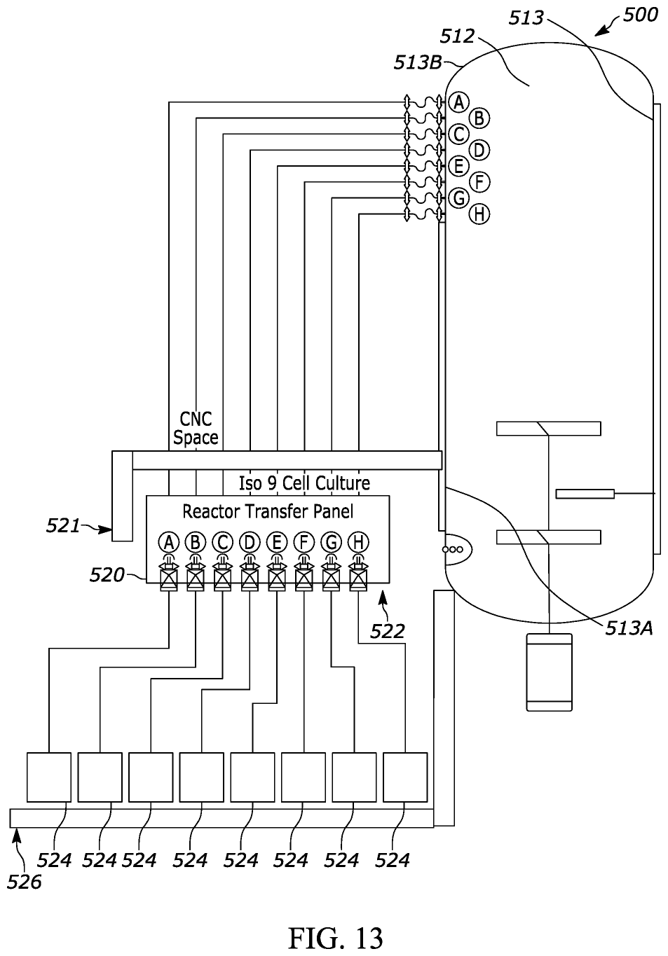

Referring now to FIG. 13, another schematic diagram of another large scale bioreactor system 500 of the present disclosure is depicted. The large scale bioreactor system 500 includes a stainless steel large scale bioreactor 512 having a side 513 with a bottom end 513A and a top end 513B. In addition, at least one stainless steel transfer panel 520 having a plurality of inputs 522 is coupled to the side 513 of the stainless steel large scale bioreactor 512 at the top end 513B of the side 513. Further, a plurality of single use feed containers 524 is coupled to one or more inputs 522 of the plurality of inputs 522 of the transfer panel 520 at a working level 526 of the stainless steel large scale bioreactor 512. While one stainless steel transfer panel 520 is depicted in FIG. 13, it will be appreciated that multiple stainless steel transfer panels may alternatively and/or additionally be coupled to the bioreactor 512 and still fall within the scope of the present disclosure. So configured, this prevents a user from having to climb a stairwell every time they connect something to the bioreactor 512. In addition, it allows making connections to single use feed containers inside a smaller clean space without locating the entire bioreactor 512 inside the clean space, for example, with some portion of the bioreactor located in a controlled, non-classified (CNC) space. This also enables users to work from a clean controlled space, operating the bioreactor 512 from the clean controlled space without having to access the top end of the bioreactor 512.

In addition, it will be appreciated that all components of any one or more of the foregoing bioreactor systems 10, 100, 200, 300 and 500 needing steaming may be steamed while the associated bioreactor 12, 112, 212, 312, 512 is being steamed. In this scenario, the steam source is from the bioreactor tank 12, 112, 312, 412, 512 and steam flows out through the valves and to the steam traps depicted in the figures. For example, in the bioreactor system 10 of FIG. 2, steam may flow out of the bioreactor 12 through the first and second valves 16, 18 to the third and fourth valves 20, 21 and to the steam trap 22. Likewise, in the bioreactor system 100 of FIG. 6, steam may also flow out of the bioreactor 112, through the first, second, third, and fourth valves 116, 118, 120, 121 and to the steam trap 122. In a similar manner, in the bioreactor system 200 of FIG. 9, steam may again flow out of the bioreactor 212 into the first, second, third and fourth valves 216, 218, 220, 221 and to the steam trap 222. Similarly, in the bioreactor system 300, the steam may again flow from the bioreactor 312 and into the first and second valves 316, 318, through the third and fourth valves 320, 321, and to the stream trap 322.

In view of the foregoing, it will be appreciated that any one or more the foregoing bioreactors 12, 112, 212, 312 may be integrated with any one or more of the foregoing perfusion devices, such as the reusable perfusion devices or single use perfusion devices 36, 136, 236 according to one or more of the following methods. Specifically, and according to one example, a method of integrating at least one single use perfusion device 36, 136, 236 with a stainless steel large scale bioreactor 12, 112, 212, 312 comprises coupling one of: (1) a connector assembly 26, such as an aseptic connector assembly 26 to at least one valve assembly 14, 214 of the stainless steel large scale bioreactor 12, 112, 212, 312; or (2) an autoclaved valve assembly 116 to a side 113 of the stainless steel large scale bioreactor 112. In addition, the method further comprises coupling one of: (1) an autoclaved valve assembly 40, 240 of an ATF assembly of a single use perfusion device 36, 236 to the connector assembly 26; or (2) an irradiated perfusion device 136 to the autoclaved valve assembly. Moreover, the method still further comprises managing pressure of the perfusion device 36, 136, 236 via at least one pressure sensor of the perfusion device 36, 136, 236 and automatically reducing one or more of a flow rate or a pressure in the perfusion device 36, 136, 236 by a control system 190 upon detecting a pressure greater than a safe limit pressure.

In one example, the method comprises coupling the aseptic connector assembly to the at least one valve assembly 14, 214 of the stainless steel large scale bioreactor 12, 212 and coupling the autoclaved valve assembly 40 to the aseptic connector assembly 26 wherein the aseptic connector assembly 26, includes one of the triclamp connector assembly or a hose assembly. In another example, the method comprises coupling the autoclaved valve assembly 114 to the side 113 of the stainless steel large scale bioreactor 112 and coupling the perfusion device 136 to one of a plurality of the autoclaved valve assemblies 114. While only one autoclaved valve assembly 114 is depicted, it will be understood that two or more autoclaved valve assemblies 114 may be coupled to the perfusion device 136 and still fall within the scope of the present disclosure.

In yet another example, the method comprises coupling a connector assembly 360 to at least one valve assembly 314 of a stainless steel large scale bioreactor 312, the connector assembly 360 including a wye connector assembly. In addition, the method further comprises coupling the autoclaved valve assembly 40 of an ATF assembly of at least one perfusion device 36 to the connector assembly 360, wherein the at least one perfusion device 36 or feed containers 373, 524 comprise a plurality of perfusion devices or feed containers connected to the wye connector assembly. This enables multiple perfusion devices or feed containers to be coupled to the bioreactor 12, 112, 212, 312 without having to steam-in-place the bioreactor 12, 112, 212, 312 upon coupling the multiple perfusion devices or feed containers.

The above description describes various bioreactor systems and methods of integrating at least one single use perfusion device and/or at least one feed container with a stainless steel large scale bioreactor. It will be appreciated the systems and methods of the present disclosure include several advantages. For example, the systems and methods described enable connection of perfusion devices to large scale bioreactors (e.g., bioreactors with a capacity of greater than 2,000 L), replacement of a perfusion device or a feed container during a cell culture run, and pressure management for perfusion devices, all while creating a steam sterilized aseptic environment. In addition, the same valve assembly at the bioreactor enables both connection of a factory assembled and irradiated single use perfusion device and an autoclaved perfusion device and cleaning-in-place of the valve assembly of the bioreactor, without the use of an aseptic connector, for example.

Although the foregoing systems and methods, and elements thereof, have been described in terms of exemplary embodiments, they are not limited thereto. The detailed description is to be construed as exemplary only and does not describe every possible embodiment of the invention because describing every possible embodiment would be impractical, if not impossible. Numerous alternative embodiments could be implemented, using either current technology or technology developed after the filing date of this patent that would still fall within the scope of the claims defining the invention.

It should be understood that the legal scope of the invention is defined by the words of the claims set forth at the end of this patent. The appended claims should be construed broadly to include other variants and embodiments of same, which may be made by those skilled in the art without departing from the scope and range of equivalents of the devices, systems, methods, and their elements.

1. A large scale bioreactor system comprising:
a stainless steel large scale bioreactor having at least one valve assembly;
an aseptic connector assembly coupled to the least one valve assembly of the bioreactor; and
a perfusion device or reusable Alternating Tangential Filtration (ATF) assembly with an autoclaved valve assembly coupled to the aseptic connector assembly, the aseptic connector assembly including one of a triclamp aseptic connector or a hose assembly.

2. The bioreactor system of claim 1, wherein the perfusion device is a single use perfusion device, the single use perfusion device further comprising at least one pressure sensor, and the bioreactor system further comprises a control system coupled to the single use perfusion device for monitoring pressure of the single use perfusion device via the at least one pressure sensor.

3. The bioreactor system of claim 1, wherein the perfusion device or reusable ATF assembly is a single use perfusion device or reusable ATF assembly further comprising at least one pressure sensor, and the bioreactor system further comprises a control system coupled to the single use perfusion device or the reusable ATF assembly for monitoring pressure of the single use perfusion device or the reusable ATF assembly via the at least one pressure sensor.

4. The bioreactor system of claim 2, wherein the control system further comprises an alarm, the alarm configured to be activated when the control system detects a pressure greater than a safe pressure limit, the control system configured to automatically reduce one or more of a flow rate or a pressure in the single use perfusion device or the reusable ATF assembly in response to detection of the pressure greater than the safe pressure limit.

5. The bioreactor system of claim 1, wherein the bioreactor has a body with a side having a top end, wherein one or more of the at least one valve assembly of the bioreactor and the autoclaved valve assembly is coupled to the side of the bioreactor near the top portion of the bioreactor.

6. The bioreactor system of claim 1, the bioreactor having a body and a bottom portion, wherein the bioreactor is configured to be at least partially disposed in a pit and one or more of the at least one valve assembly of the stainless steel large scale bioreactor and the autoclaved valve assembly is coupled against a bottom weld seam of the bottom portion of the bioreactor.

7. The bioreactor system of claim 1, wherein the aseptic connector assembly is one of: (1) an aseptic connector valve assembly having a triclamp aseptic connector, enabling a new perfusion device or ATF assembly to be coupled to the stainless steel large scale bioreactor while the bioreactor is running a cell culture by repeating a steam-in-place of the aseptic connector assembly; or (2) a hose assembly, the hose assembly coupling the at least one valve assembly of the bioreactor to the perfusion device or ATF assembly, wherein the hose assembly comprises a hose body having a first end and a second end, the first end operatively coupled to the at least one valve assembly and the second end operatively coupled to the perfusion device or ATF assembly.

8. (canceled)

9. A large scale bioreactor system comprising:
a stainless steel large scale bioreactor having a side;
an autoclaved valve assembly coupled to the side of the bioreactor;
at least one aseptic connector coupled to the autoclaved valve assembly; and
an irradiated single use perfusion device coupled to the at least one aseptic connector.

10. The bioreactor system of claim 9, wherein the autoclaved valve assembly is a first valve assembly and the bioreactor system further comprises a second autoclaved valve assembly configured to be coupled to the side of the bioreactor adjacent to the first valve assembly, and at least one aseptic connector configured to be coupled to the second autoclaved valve assembly.

11. The bioreactor system of claim 9, wherein the irradiated single use perfusion device further comprises at least one pressure sensor, and the bioreactor system further comprises a control system coupled to the irradiated single use perfusion device for monitoring pressure of the single use perfusion device via the at least one pressure sensor.

12. The bioreactor system of claim 11, wherein the control system further comprises an alarm, the alarm configured to be activated when the control system detects a pressure greater than a safe pressure limit, the control system configured to automatically reduce one or more of a flow rate or a pressure in the single use perfusion device in response to detection of the pressure greater than the safe pressure limit.

13. The bioreactor system of claim 9, wherein the bioreactor has at least one of: (1) a body with a side having a top end, wherein the autoclaved valve assembly is coupled to the side of the bioreactor near the top end of the bioreactor; or (2) a body and a bottom end, wherein the bioreactor is configured to be at least Partially disposed in a pit and the autoclaved valve assembly is coupled against a bottom weld seam of the bottom portion of the bioreactor.

14. (canceled)

15. A large scale bioreactor system comprising:
a stainless steel large scale bioreactor having at least one valve assembly;
a single use adapter assembly including a wye connector assembly coupled to the at least one valve assembly; and
a plurality of single use perfusion devices connected to the single use adapter assembly, enabling multiple perfusion units to be coupled to the bioreactor without having to steam-in-place the bioreactor upon coupling multiple single use perfusion units.

16. The bioreactor system of claim 15, further comprising a single use manifold coupled to one of the single use adapter assembly or the at least one valve assembly, the single use manifold including any one of two, three, four, five, six, seven, or eight arms, each arm including an inlet for coupling to a single use perfusion unit, enabling the plurality of single use perfusion devices to be operatively coupled to the stainless steel large scale bioreactor.

17. The bioreactor system of claim 15, wherein the wye connector assembly is a first wye connector assembly, and a plurality of wye connector assemblies are coupled to the first wye connector assembly, enabling the plurality of single use perfusion devices to be operatively coupled to the stainless steel large scale bioreactor.

18. The bioreactor system of claim 15, wherein the single use perfusion device further comprises at least one pressure sensor, and the bioreactor system further comprises a control system for monitoring pressure of at least one single use perfusion device of the plurality of single use perfusion devices via the at least one pressure sensor.

19. The bioreactor system of claim 18, wherein the control system further comprises an alarm, the alarm configured to be activated when the control system detects a pressure greater than a safe pressure limit, the control system configured to automatically reduce one or more of a flow rate or a pressure in the at least one single use perfusion device of the plurality of single use pressure devices in response to detection of the pressure greater than the safe pressure limit.

20. The bioreactor system of claim 15, wherein the bioreactor has a body with a side having a top end, wherein the at least one valve assembly of the bioreactor is coupled to the side of the bioreactor near the top portion of the bioreactor.

21. The bioreactor system of claim 15, wherein the bioreactor has a body and a bottom portion, wherein the bioreactor is configured to be at least partially disposed in a pit and the at least one valve assembly of the stainless steel large scale bioreactor is coupled against a bottom weld seam of the bottom portion of the bioreactor.

22. A large scale bioreactor system comprising:
a stainless steel large scale bioreactor;
at least one stainless steel transfer panel having a plurality of inputs coupled to the bioreactor; and
a plurality of feed containers coupled to the at least one stainless steel transfer panel at a working level of the bioreactor.

23. The bioreactor system of claim 22, wherein the at least one stainless steel transfer panel is a first stainless steel transfer panel, and wherein the bioreactor system further comprises one or more additional stainless steel transfer panels, each of which is configured to be coupled to additional, multiple feed containers.

24. The bioreactor system of claim 22, wherein the plurality of feed containers further comprises at least one pressure sensor, and the bioreactor system further comprises a control system for monitoring pressure of at least one feed container of the plurality of single use feed containers via the at least one pressure sensor, wherein the at least one feed container is at least one single use feed container.

25. The bioreactor system of claim 24, wherein the control system further comprises an alarm, the alarm configured to be activated when the control system detects a pressure greater than a safe pressure limit, the control system configured to automatically reduce one or more of a flow rate or a pressure in the at least one single use feed container of the plurality of single use feed containers in response to detection of the pressure greater than the safe pressure limit.

26. The bioreactor system of claim 22, wherein the bioreactor has at least one of: (1) a body with a side having a top end and at least one valve assembly that is coupled to the side of the bioreactor near the top end of the bioreactor; or (2) at least one valve assembly and a body with a bottom portion, wherein the bioreactor is configured to be at least partially disposed in a pit and the at least one valve assembly of the stainless steel large scale bioreactor is coupled against a bottom weld seam of the bottom portion of the bioreactor.

27. (canceled)

28. The bioreactor system of claim 1, wherein the stainless steel large scale bioreactor has one of: (1) a volume of greater than 2,000 L; or (2) a volume in the range of greater than 2,000 L to 20,000 L.

29. (canceled)

30. A method of integrating at least one perfusion device with a stainless steel large scale bioreactor, the method comprising:
coupling one of: (1) a connector assembly to at least one valve assembly of a stainless steel large scale bioreactor; or (2) an autoclaved valve assembly to a side of the stainless steel large scale bioreactor;
coupling one of: (1) an autoclaved valve assembly of an Alternating Tangential Filtration (ATF) assembly of a perfusion device to the connector assembly; or (2) an irradiated perfusion device to the autoclaved valve assembly; and
managing pressure of the perfusion device via at least one pressure sensor of the perfusion device and automatically reducing one or more of a flow rate or a pressure in the perfusion device upon detecting a pressure greater than a safe limit pressure by a control system.

31. The method of claim 30, wherein coupling one of: (1) a connector assembly to at least one valve assembly of a stainless steel large scale bioreactor; or (2) an autoclaved valve assembly to a side of the stainless steel large scale bioreactor; comprises coupling an aseptic connector valve assembly to the at least one valve assembly of the stainless steel large scale bioreactor.

32. The method of claim 31, wherein coupling one of: (1) an autoclaved valve assembly of an ATF assembly of a perfusion device to the connector assembly; or (2) an irradiated perfusion device to the autoclaved assembly comprises coupling the autoclaved valve assembly of the perfusion device to the connector assembly, the connector assembly comprising one of a triclamp connector assembly or a hose assembly.

33. The method of claim 32, wherein coupling one of: (1) a connector assembly to at least one valve assembly of a stainless steel large scale bioreactor; or (2) an autoclaved valve assembly to a side of the stainless steel large scale bioreactor comprises coupling an autoclaved assembly to the side of the stainless steel large scale bioreactor.

34. The method of claim 33, wherein coupling one of: (1) an autoclaved valve assembly of an ATF assembly of a perfusion device to the connector assembly; or (2) an irradiated perfusion device to the autoclaved valve assembly comprises coupling the irradiated perfusion device to the autoclaved assembly.

35. The method of claim 30, wherein coupling one of: (1) a connector assembly to at least one valve assembly of a stainless steel large scale bioreactor; or (2) an autoclaved valve assembly to a side of the stainless steel large scale bioreactor comprises coupling a connector assembly to at least one valve assembly of a stainless steel large scale bioreactor, the connector assembly including a wye connector assembly.

36. The method of claim 35, wherein coupling one of: (1) an autoclaved valve assembly of an ATF assembly of at least one perfusion device to the connector assembly; or (2) an irradiated perfusion device to at least one aseptic connector comprises coupling the autoclaved valve assembly of an ATF assembly of at least one perfusion device to the connector assembly, wherein the at least one perfusion device comprises a plurality of perfusion devices connected to the wye connector assembly, enabling multiple perfusion devices to be coupled to the bioreactor without having to steam-in-place the bioreactor upon coupling multiple perfusion devices.

37. The method of claim 30, further comprising at least one of: (1) coupling at least one stainless steel transfer panel having a plurality of inputs to the stainless steel large scale bioreactor; or (2) coupling one or more of the at least one valve assembly of the stainless steel large scale bioreactor and the autoclaved valve assembly to a side of the stainless steel large scale bioreactor.

38. (canceled)

39. The method of claim 30, wherein the stainless steel large scale bioreactor has one of: (1) a volume of greater than 2,000 L; or (2) a volume in the range of greater than 2,000 L to 20,000 L.

40. (canceled)
